(19) United States
(12) Patent Application Publication
(10) Pub. No.: US 2010/0305143 A1
(43) Pub. Date: Dec. 2, 2010

(54) PYRROLOPYRIMIDINE COMPOUNDS

(75) Inventors: Allan Jordan, Berkshire (GB); Simon Bedford, Berkshire (GB); Klenke Burkhard, Berkshire (GB); Ian Yule, Berkshire (GB); Karinne Poullennec, Berkshire (GB)

Correspondence Address: BANNER & WITCOFF, LTD., 1100 13th STREET, N.W., SUITE 1200, WASHINGTON, DC, 20005-4051, US

(73) Assignee: VERNALIS (R & D) LTD., Berkshire (GB)

(21) Appl. No.: 12/677,919

(22) PCT Filed: Sep. 19, 2008
(86) PCT No.: PCT/GB2008/003179

§ 371 Date: Aug. 16, 2010

(30) Foreign Application Priority Data
Sep. 21, 2007 (GB) 0718433.6

(51) Int. Cl. A61K 31/519; C07D 487/04; A61P 29/00; A61P 3/10; A61P 35/00; A61P 11/06; A61P 11/00

(52) U.S. Cl. 514/265.1; 544/280

(57) ABSTRACT

Compounds of formula (I) are A_{2B} receptor antagonists: wherein Ri is optionally substituted aryl or an optionally substituted monocyclic heteroaryl group having 5 or 6 ring atoms; R₂ and R₃ are independently selected from hydrogen, or optionally substituted C₁-C₆ alkyl, C₁-C₆ alkoxy-(C₁-C₆)-alkyl, C₃-C₈ cycloalkyl, aryl, heteroaryl, aryl-(C₁-C₆)-alkyl, or heteroaryl-(C₁-C₆)-alkyl; R₄ and R₅ are independently selected from hydrogen, optionally substituted C₁-C₆alkyl, optionally substituted aryl, aryl-(C₁-C₆)-alkyl optionally substituted in the ring part thereof, —NHR₇—N(—R₈)—R₉, —NH—(C═O)—R₁₀, —(C═O)—NH—R₁₁, —(C═O)—O—R₁₂, or halo; R₆ is hydrogen, C₁-C₆ alkyl, aryl-(C₁-C₆)-alkyl, —(C═O)—NH—R₁₃, —(C═O)—R₁₄, aryl, heteroaryl, hydroxy-(C₁-C₆)-alkyl, or C₃-C₈ cycloalkyl-alkyl; and R₇, R₈, R₉, R₁₀, R₁₁, R₁₂, R₁₃ and R₁₄ are independently selected from C₁-C₆ alkyl, aryl, aryl-(C₁-C₆)-alkyl and heteroaryl.

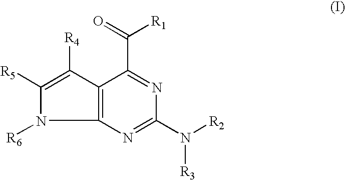

This invention relates to novel pyrrolopyrimidine derivatives having A_{2B} receptor antagonistic activity, to the use of such compounds in medicine, in relation to the treatment of disorders which are responsive to antagonism of the A_{2B} receptor such as nociception, asthma, COPD, inflammatory disorders, diabetes, diabetic retinopathy and cancer, and to pharmaceutical compositions containing such compounds.

### BACKGROUND TO THE INVENTION

Adenosine is a naturally occurring purine nucleoside, the effects of which include stimulation of nociception afferents, bronchconstriction, immunosupression, vasodilation, inhibition of platelet aggregation, cardiac depression and inhibition of neurotransmitter release.

Adenosine produces a wide range of pharmacological effects mediated by activation of specific cell surface receptors, which are members of the G-protein coupled receptor family. Four subtypes of adenosine receptors have been identified, designated A₁, A_{2A}, A_{2B} and A₃.

The A_{2B} adenosine receptor subtype is coupled to the G_{S} G-protein and stimulates adenylyl cyclase activity. Although significant advancement has been made in the understanding of the molecular pharmacology and physiology of A_{2B} adenosine receptors, due to the lack of highly potent and selective ligands for this receptor subtype, many questions about the patho-physiological role of A_{2B} receptors are yet to be resolved (Feoktistov and Biaggioni, Pharmacological Reviews (1997), 49(4), 381-402).

A_{2B} receptors have been implicated in:
(i) the regulation of mast cell secretion (Feoktistov and Biaggioni., Journal of Clinical Investigation (1995), 96(4), 1979-86).
(ii) pain (Abo-Salem et al., Journal of Pharmacology and Experimental Therapeutics (2004), 308(1), 358-366.).
(iii) inflammation (Yang et al., Journal of Clinical Investigation (2006), 116(7), 1913-1923).
(iv) cancer (Zeng et al., Drug Development Research (2003), 58(4), 405-411).
(v) diabetes (Harada et al., Journal of Medicinal Chemistry (2001), 44(2), 170-179).
(vi) gene expression (Boyle et al., Arthritis & Rheumatism (1996), 39(6), 923-930).
(vii) cell growth (Dubey et al., Hypertension (1996), 27(3 Pt 2), 786-93 Hypertension (1996), 27(3 Pt 2), 786-93, Dubey et al., Hypertension (1998), 31(1 Pt 2), 516-21).
(viii) intestinal functions (Murthy et al., Journal of Neurochemistry, (1995), 64(1), 77-84).
(ix) neurosecretion (Mateo et al., 1995).
(x) vascular tone (Haynes et al., American Journal of Physiology (1995), 268(5, Pt. 2), H1862-H1868).
(xi) asthma (Feoktistov et al., Trends in pharmacological sciences (1998), 19(4), 148-153; Holgate, British Journal of Pharmacology (2005), 145(8), 1009-1015).
(xii) COPD (Van den Berge et al., Drugs in R&D (2007), 8(1), 13-23).

Thus, there remains a medical need for low molecular weight A_{2B} antagonists with pharmacokinetic and pharmacodynamic properties making them suitable for use as pharmaceutical agents. There also remains a medical need for new treatments of disorders mediated by the A_{2B} receptor, particularly nociception, asthma, COPD, inflammatory disorders, diabetes, diabetic retinopathy and cancer. The object of the present invention is to provide such pharmaceutical agents and treatments.

It has now been found that certain pyrrolopyrimidine derivatives show efficacy as A_{2B} antagonists.

### BRIEF DESCRIPTION OF THE INVENTION

The present invention relates to a class of substituted pyrrolopyrimidine compounds useful as A_{2B} antagonists, for example, for the treatment of nociception, asthma, COPD, inflammatory disorders, diabetes, diabetic retinopathy and cancer. A core pyrrolo-pyrimidine bicyclic ring, with substitution on the pyrimidine portion by a (hetero)arylcarbonyl group in addition to an amino group are principle characterising features of the compounds with which the invention is concerned.

### DETAILED DESCRIPTION OF THE INVENTION

According to the present invention, there is provided a compound of formula (I) or a pharmaceutically acceptable salt, hydrate or solvate thereof:

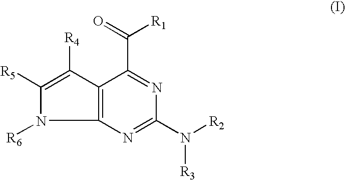

wherein

R₁ is optionally substituted aryl or an optionally substituted monocyclic heteroaryl group having 5 or 6 ring atoms;

R₂ and R₃ are independently selected from hydrogen, or optionally substituted C₁-C₆ alkyl, C₁-C₆ alkoxy-(C₁-C₆)-alkyl, C₃-C₈ cycloalkyl, aryl, heteroaryl, aryl-(C₁-C₆)-alkyl, or heteroaryl-(C₁-C₆)-alkyl;

R₄ and R₅ are independently selected from hydrogen, optionally substituted C₁-C₆ alkyl, optionally substituted aryl, aryl-(C₁-C₆)-alkyl optionally substituted in the ring part thereof, —NHR₇, —N(—R₈)—R₉, —NH—(C═O)—R₁₀, —(C═O)—NH—R₁₁, —(C═O)—O—R₁₂, or halo;

R₆ is hydrogen, C₁-C₆ alkyl, aryl-(C₁-C₆)-alkyl, —(C═O)—NH—R₁₃, —(C═O)—R₁₄, aryl, heteroaryl, hydroxy-(C₁-C₆)-alkyl, or C₃-C₈ cycloalkyl-alkyl; and

R₇, R₈, R₉, R₁₀, R₁₁, R₁₂, R₁₃ and R₁₄ are independently selected from C₁-C₆ alkyl, aryl, aryl-(C₁-C₆)-alkyl and heteroaryl.

The active compounds of formula (I) are antagonists of the A_{2B} receptor and are useful for the treatment, prevention and suppression of disorders mediated by the A_{2B} receptor. Such disorders include nociception; asthma; chronic obstructive pulmonary disease (COPD); inflammatory diseases such as rheumatoid arthritis, multiple sclerosis, lupus, psoriasis and inflammatory bowel disease; diabetes mellitus or diabetes insipidus; diabetic retinopathy and cancer.

According to a further embodiment of the present invention there is provided the use of a compound of formula (I), or a pharmaceutically acceptable salt, hydrate, solvate, or prodrug thereof, in the manufacture of a medicament for the treatment of disorders mediated by the adenosine A_{2B} receptor.

According to a further embodiment of the present invention there is provided a method of treatment of a disorder mediated by the A_{2B} receptor comprising administration to a subject in need of such treatment an effective dose of the compound of formula (I), or a pharmaceutically acceptable salt, hydrate, solvate, or prodrug thereof.

According to a further embodiment of the present invention there is provided a pharmaceutical composition comprising a compound of formula (I), or a pharmaceutically acceptable salt, hydrate, solvate, or prodrug thereof, and a pharmaceutically acceptable carrier.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “divalent (Cₐ-C_{b})alkenylene radical” refers to a hydrocarbon chain having from a to b carbon atoms, at least one double bond, and two unsatisfied valences.

As used herein the term “cycloalkyl” refers to a saturated carbocyclic radical having from 3-8 carbon atoms and includes, for example, cyclopropyl, cyclobutyl, cyclopentyl, cyclohexyl, cycloheptyl and cyclooctyl.

As used herein the term “cycloalkenyl” refers to a carbocyclic radical having from 3-8 carbon atoms containing at least one double bond, and includes, for example, cyclopentenyl, cyclohexenyl, cycloheptenyl and cyclooctenyl.

As used herein the term “carbocyclic” refers to a mono- or bi-cyclic radical whose ring atoms are all carbon, and includes monocyclic aryl, cycloalkyl, and cycloalkenyl radicals, provided that no single ring present has more than 8 ring members. A “carbocyclic” group includes a mono-bridged or multiply-bridged cyclic alkyl group.

As used herein the term “aryl” refers to a mono-, bi- or tri-cyclic carbocyclic aromatic radical. Illustrative of such radicals are phenyl, biphenyl and napthyl.

As used herein the term “heteroaryl” refers to a mono-, bi- or tri-cyclic aromatic radical containing one or more heteroatoms selected from S, N and O. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in particular refers to a mono-, bi- or tri-cyclic non-aromatic radical containing one or more heteroatoms selected from S, N and O, to groups consisting of a monocyclic non-aromatic radical containing one or more such heteroatoms which is covalently linked to another such radical or to a monocyclic carbocyclic radical, and to a mono-, bi- or tri-cyclic non-aromatic radical containing one or more heteroatoms selected from S, N and O which is mono-bridged or multiply-bridged. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with at least one substituent, for example selected from (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, halo (including fluoro and chloro), trifluoromethyl, trifluoromethoxy, nitro, nitrile (—CN), oxo, phenyl, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl group, or R^{A} and R^{B} when attached to the same nitrogen may form a cyclic amino ring such as a morpholinyl, piperidinyl or piperazinyl ring. An “optional substituent” or “substituent” may be one of the foregoing substituent groups.

As used herein the term “salt” includes base addition, acid addition and quaternary salts. Compounds of the invention which are acidic can form salts, including pharmaceutically or veterinarily acceptable salts, with bases such as alkali metal hydroxides, e.g. sodium and potassium hydroxides; alkaline earth metal hydroxides e.g. calcium, barium and magnesium hydroxides; with organic bases e.g. N-ethyl piperidine, dibenzylamine and the like. Those compounds (I) which are basic can form salts, including pharmaceutically or veterinarily acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic and p-toluene sulphonic acids and the like.

For a review on suitable salts, see Handbook of Pharmaceutical Salts: Properties, Selection, and Use by Stahl and Wermuth (Wiley-VCH, Weinheim, Germany, 2002).

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Compounds with which the invention is concerned which may exist in one or more stereoisomeric form, because of the presence of asymmetric atoms or rotational restrictions, can exist as a number of stereoisomers with R or S stereochemistry at each chiral centre or as atropisomeres with R or S stereochemistry at each chiral axis. The invention includes all such enantiomers and diastereoisomers and mixtures thereof.

So-called ‘pro-drugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Also included within the scope of the invention are metabolites of compounds of formula (I), that is, compounds formed in vivo upon administration of the drug. Some examples of metabolites include

(i) where the compound of formula (I) contains a methyl group, an hydroxymethyl derivative thereof (—CH₃—>—CH₂OH):
(ii) where the compound of formula (I) contains an alkoxy group, an hydroxy derivative thereof (—OR—>—OH);
(iii) where the compound of formula (I) contains a tertiary amino group, a secondary amino derivative thereof (—NR¹R²—>—NHR¹ or —NHR²);
(iv) where the compound of formula (I) contains a secondary amino group, a primary derivative thereof (—NHR¹—>—NH₂);
(v) where the compound of formula (I) contains a phenyl moiety, a phenol derivative thereof (-Ph->-PhOH); and
(vi) where the compound of formula (I) contains an amide group, a carboxylic acid derivative thereof (—CONH₂—>COOH).

### The Group R₁

In the compounds in accordance with the invention, R₁ is selected from optionally substituted aryl or an optionally substituted monocyclic heteroaryl group having 5 or 6 ring atoms.

In a subclass of compounds with which the invention is concerned, R₁ is optionally substituted phenyl, preferably phenyl.

In a further subclass of compounds with which the invention is concerned, R₁ is an optionally substituted monocyclic heteroaryl group having 5 ring atoms.

Presently, it is preferred that R₁ is optionally substituted thienyl. Particularly preferred for R₁ is 2-thienyl or 3-methyl-thien-2-yl.

### The Groups R₂ and R₃

In the compounds in accordance with the invention, R₂ and R₃ are independently selected from hydrogen, or optionally substituted C₁-C₆ alkyl, C₁-C₆ alkoxy-(C₁-C₆)-alkyl, C₃-C₈ cycloalkyl, aryl, heteroaryl, aryl-(C₁-C₆)-alkyl, or heteroaryl-(C₁-C₆)-alkyl.

In a subclass of compounds with which the invention is concerned, R₂ is hydrogen and R₃ is heteroaryl-(C₁-C₆)-alkyl. In such cases, heteroaryl may be an optionally substituted monocyclic heteroaryl group having 5 or 6 ring atoms. Preferred heteroaryl rings include furan, thiophene, pyrrole or pyrimidine. Methyl and ethyl are preferred for C₁-C₆-alkyl.

Presently, it is particularly preferred that R₂ is hydrogen and R₃ is 2-pyridylmethyl, 3-pyridylmethyl, 4-pyridylmethyl, or 3-pyridyl-1-ethyl.

### The Groups R₄ and R₅

In the compounds in accordance with the invention, R₄ and R₅ are independently selected from hydrogen, optionally substituted C₁-C₆ alkyl, optionally substituted aryl, aryl-(C₁-C₆)-alkyl optionally substituted in the ring part thereof, —NHR₇, —N(—R₈)—R₉, —NH—(C═O)—R₁₀, —(C═O)—NH—R₁₁, —(C═O)—O—R₁₂, or halo; wherein R₇, R₈, R₉, R₁₀, R₁₁, and R₁₂ are independently selected from C₁-C₆ alkyl, aryl, aryl-(C₁-C₆)-alkyl and heteroaryl.

In a subclass of compounds with which the invention is concerned, R₄ and R₅ are independently selected from hydrogen, halo, optionally substituted aryl, or heteroarylcarbonylamino. In such cases halo may be represented by fluoro, chloro or bromo; aryl includes phenyl; and heteroaryl may be an optionally substituted monocyclic heteroaryl group having 5 or 6 ring atoms. Preferred heteroaryl rings include furan, thiophene, pyrrole or pyrimidine.

It is presently preferred that R₄ and R₅ are independently selected from hydrogen, chloro, bromo, phenyl, 2-methoxyphenyl, 3-methoxyphenyl, 4-methoxyphenyl, 4-methylsulphonylphenyl, or 2-thienylcarbonylamino.

In another subclass of compounds with which the invention is concerned R₅ is —N(—R₁₅)—R₁₆, wherein R₁₅ and R₁₆ are independently selected from hydrogen or C₁-C₆ alkyl. It is preferred that R₅ is amino, methylamino, ethylamino, or dimethylamino.

### The Group R₆

In the compounds in accordance with the invention, R₆ is hydrogen, C₁-C₆ alkyl, aryl-(C₁-C₆)-alkyl, —(C═O)—NH—R₁₃, —(C═O)—R₁₄, aryl, heteroaryl, hydroxy-(C₁-C₆)-alkyl, or C₃-C₈ cycloalkyl-alkyl; wherein R₁₃ and R₁₄ are independently selected from C₁-C₆ alkyl, aryl, aryl-(C₁-C₆)-alkyl and heteroaryl.

In a subclass of compounds with which the invention is concerned, R₆ is hydrogen, C₁-C₆ alkyl, aryl-(C₁-C₆)-alkyl, hydroxy-(C₁-C₆)-alkyl, or C₃-C₃ cycloalkyl-alkyl.

It is presently preferred that R₆ is hydrogen, methyl, n-propyl, n-pentyl, benzyl, hydroxymethyl, or cyclopropylmethyl.

Specific compounds with which the invention is concerned include those of the Examples.

The present invention may be employed in respect of a human or animal subject, more preferably a mammal, more preferably a human subject.

As used herein, the term “treatment” as used herein includes prophylactic treatment.

The compound of formula (I) may be used in combination with one or more additional drugs useful in the treatment of the disorders mentioned above, the components being in the same formulation or in separate formulations for administration simultaneously or sequentially.

It will be understood that the specific dose level for any particular patient will depend upon a variety of factors including the activity of the specific compound employed, the age, body weight, general health, sex, diet, time of administration, route of administration, rate of excretion, drug combination and the causative mechanism and severity of the particular disease undergoing therapy. In general, a suitable dose for orally administrable formulations will usually be in the range of 0.1 to 3000 mg, once, twice or three times per day, or the equivalent daily amount administered by infusion or other routes. However, optimum dose levels and frequency of dosing will be determined by clinical trials as is conventional in the art.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

There are multiple synthetic strategies for the synthesis of the compounds (I) with which the present invention is concerned, but all rely on known chemistry, known to the synthetic organic chemist. Thus, compounds according to formula (I) can be synthesised according to procedures described in the standard literature and are well-known to the one skilled in the art. Typical literature sources are “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “Chemical Abstracts” or “Beilstein”. Such literature methods include those of the preparative Examples herein, and methods analogous thereto.

Examples of methods known in the art of organic chemistry in general, by which the compounds of the present invention may be prepared, are included in the following reaction schemes and procedures.

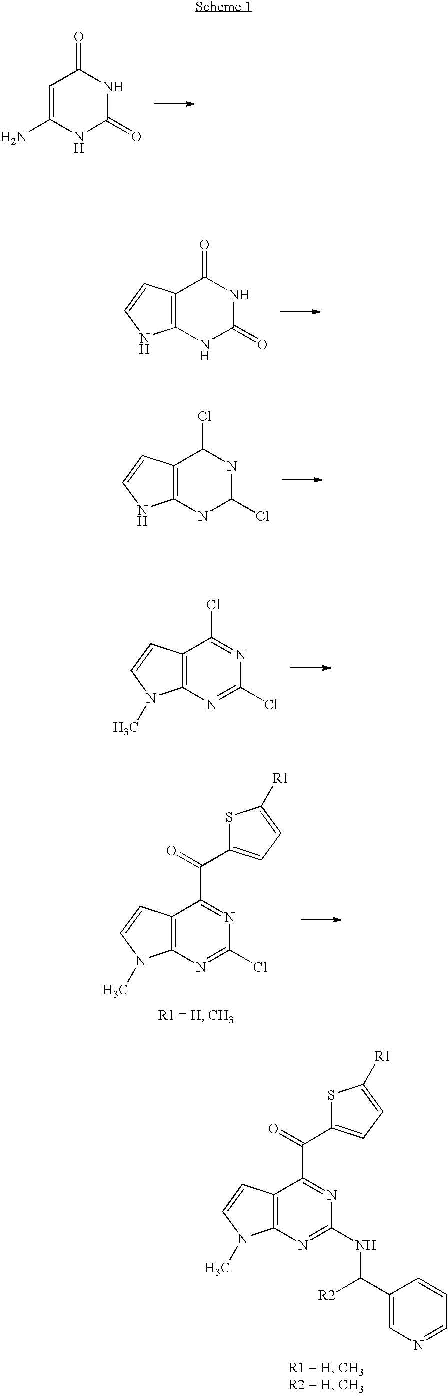

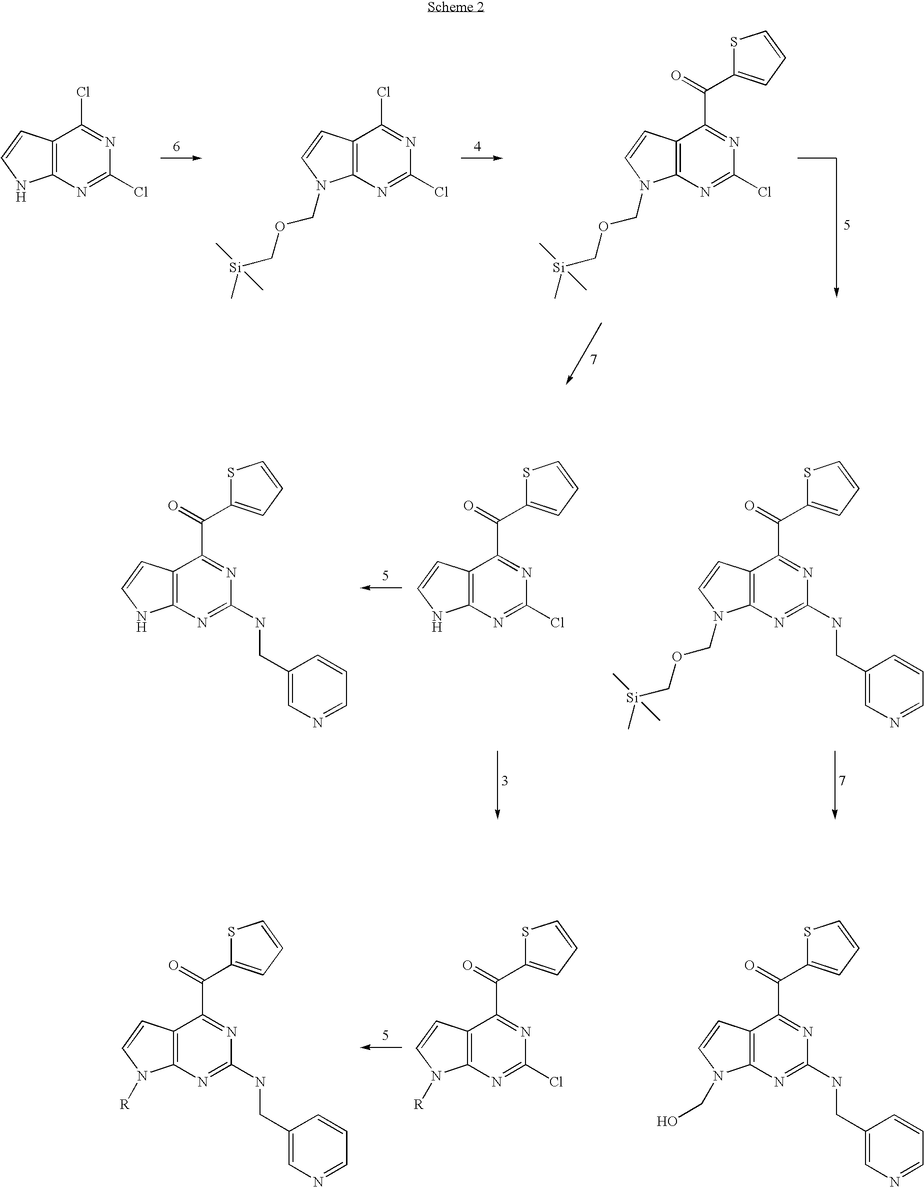

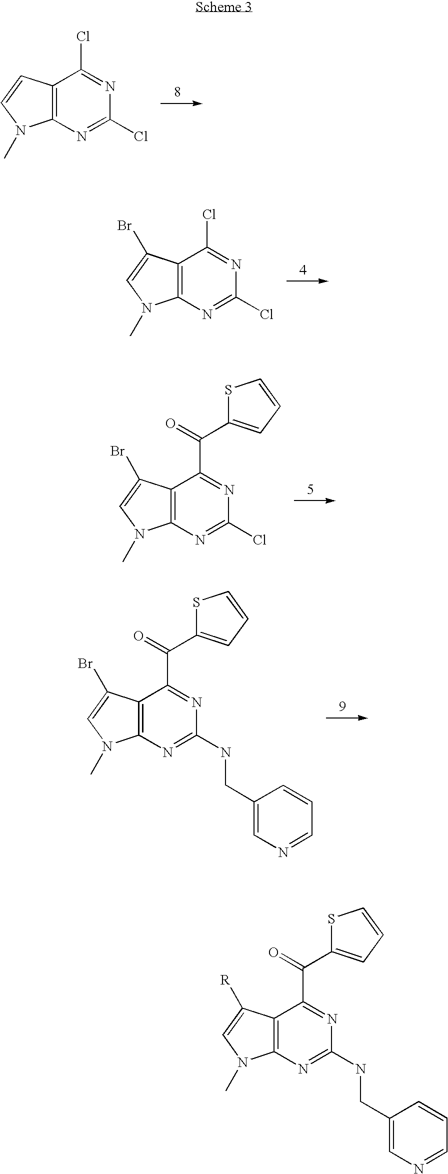

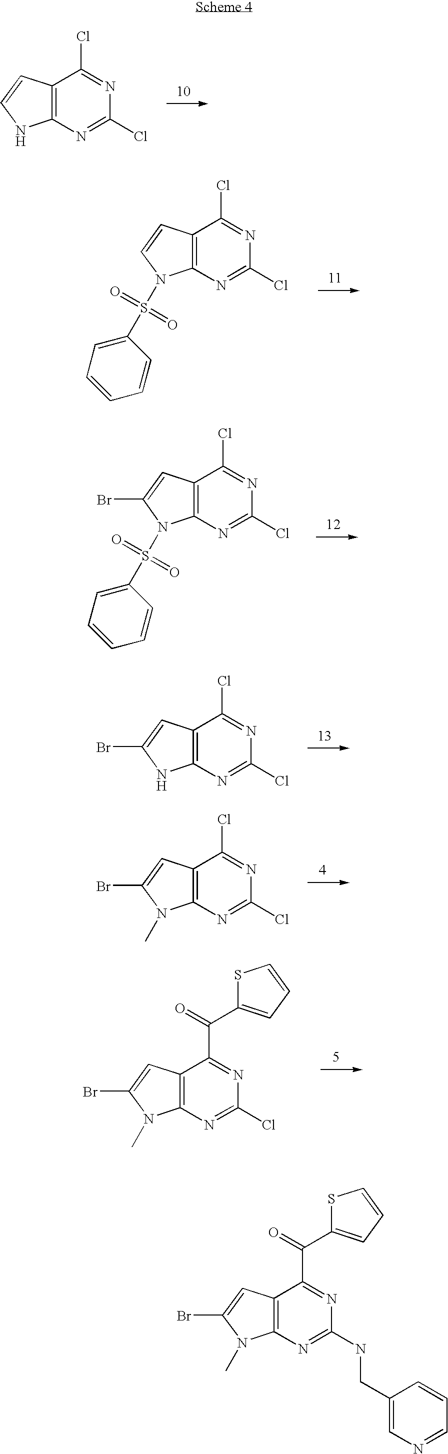

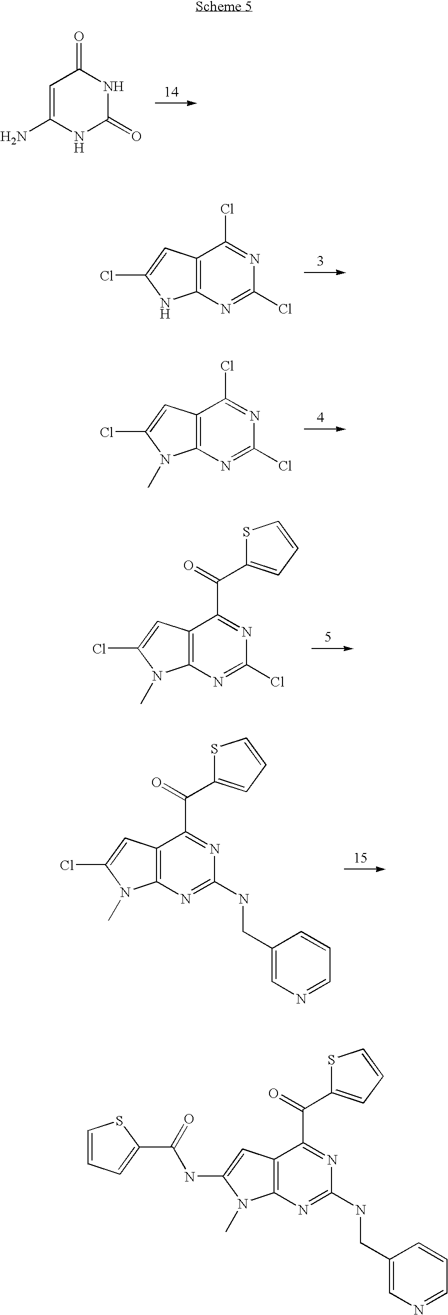

### EXAMPLES

The following examples illustrate the preparation of specific compounds of the invention and are not intended to be limiting of the full scope of the invention.

The compounds of Examples 1 to 10 were prepared as per the methodology described in scheme 1.

### Example 1

### 2,4-Dihydroxy-7H-pyrrolo[2,3-d]pyrimidine

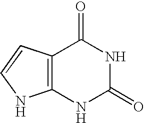

A solution of commercially available 6-amino-1H-pyrimidine-2,4-dione (25 g, 0.2 mol) and NaOAc (20 g, 0.24 mol) in 1.25 L of distilled water was treated with chloroacetaldehyde (25 mL, 50% w/w in water). After 3 h at reflux, the reaction mixture was filtered while still warm, to remove the brown solid. The yellow mother liquor was cooled to RT then acidified to pH —4 by addition of 2.5 M HCl. The desired product (9.722 g, 33%) was isolated by filtration and finally dried in the oven. ¹H NMR (d₆-DMSO) δ 11.46 (1H, br s), 11.11 (1H, br s), 10.49 (1H, br s), 6.57 (1H, dd, J=2.0, 3.6 Hz), 6.22 (1H, dd, J=2.0, 3.2 Hz).

### Example 2

### 2,4-Dichloro-7H-pyrrolo[2,3-d]pyrimidine

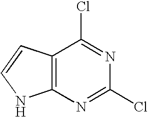

Example 1 (9.722 g, 64.329 mmol) was dissolved in 100 mL warm 1N NaOH. The water was removed in vacuo to one fourth then cooled to 0° C. The sodium salt (5.907 g, 34.123 mmol) was isolated by filtration and dried in the oven for a few hours. The sodium salt was dissolved in PhPOCl₂ (29 mL, 204.7 mmol) and stirred at 180° C. for 3 h. While still warm, the reaction mixture was poured over crushed ice. The aqueous layer was extracted with EtOAc and the organic layer was washed with sat. NaHCO₃/NaHCO₃(s), dried (MgSO₄) and filtered. After evaporation of the volatiles, the desired product (2 g, 16%) was isolated by recrystallisation from toluene. ¹H NMR (d₆-DMSO) δ 12.79 (1H, br s), 7.74 (1H, dd, J=2.0, 4.0 Hz), 6.67 (1H, J=2.3, 3.2 Hz).

### Example 3

### 2,4-Dichloro-7-methyl-7H-pyrrolo[2,3-d]pyrimidine

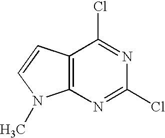

Example 2 (1 g, 5.318 mmol) and powdered KOH (446 mg, 7.978 mmol) was dissolved in 4 mL anhydrous DMSO. After 1 h at RT, the reaction mixture was quenched with water and the aqueous layer was extracted with EtOAc (2×). The organics were combined, dried (MgSO₄), filtered and the volatiles were removed in vacuo. The residue was purified by flash chromatography (SiO₂, hexanes/EtOAc, 2:1) to provide the desired product (892 mg, 83%) as a colourless solid. ¹H NMR (d₆-DMSO) δ 7.76 (1H, d, J=3.6 Hz), 6.70 (1H, d, J=3.6 Hz), 3.82 (3H, s).

### Example 4

### (2-Chloro-7-methyl-7H-pyrrolo[2,3-d]pyrimidin-4-yl)-thiophen-2-yl-methanone

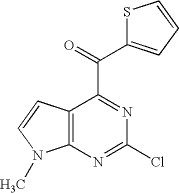

A solution of Example 3 (654 mg, 3.237 mmol), N,N-dimethylimidazolium iodide (216 mg, 0.971 mmol) and 2-thiophenecarboxaldehyde (363 μL, 3.884 mmol) in 20 mL anhydrous THF was treated with NaH (155.4 mg, 3.884 mmol) portionwise. After 30 min at RT, the reaction was quenched with water. The aqueous layer was extracted with EtOAc (2×20 mL). The organics were combined, dried (MgSO₄) and filtered. After evaporation of the volatiles, the residue was purified by flash chromatography (25 g Isolute SiO₂ cartridge, gradient hexanes 100% to hexanes/EtOAc, 2:1) to provide the desired product (736 mg, 82%) as an orange solid. ¹H NMR (CDCl₃) δ 8.50 (1H, dd, J=1.6, 3.6 Hz), 7.81 (1H, dd, J=1.2, 4.8 Hz), 7.36 (1H, d, J=3.2 Hz), 7.23 (1H, dd, J=4.0, 5.2 Hz), 7.19 (1H, d, J=3.2 Hz), 3.92 (3H, s).

### Example 5

### (2-Chloro-7-methyl-7H-pyrrolo[2,3-d]pyrimidin-4-yl)-(5-methyl-thiophen-2-yl)-methanone

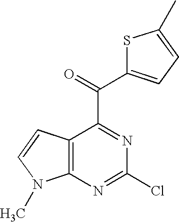

The title compound was prepared by the route outlined in scheme 1, following the same procedure described for step 4. Purification by recrytallisation from EtOAc. ¹H NMR (CDCl₃) δ 8.31 (1H, d, J=4.0 Hz), 7.33 (1H, d, J=3.6 Hz), 7.17 (1H, d, J=3.2 Hz), 6.90 (1H, dd, J=0.8, 3.2 Hz), 3.90 (3H, s), 2.60 (3H, s). LC-MS: m/z=291 [M+H]⁺; RT=3.99 min.

### Example 6

### {7-Methyl-2-[(pyridin-3-ylmethyl)-amino]-7H-pyrrolo[2,3d]pyrimidin-4-yl}-thiophen-2-yl-methanone

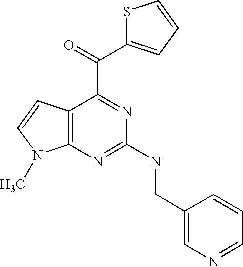

A solution of Example 4 (800 mg, 2.88 mmol) and 3-picolylamine in 30 mL n-BuOH was refluxed for 3 days. After cooling down to RT, the reaction was acidified with 2 M HCl, washed with EtOAc. The aqueous layer was basified with solid NaHCO₃ and extracted with EtOAc (2×30 mL). The organics were combined, dried (MgSO₄), filtered and the volatiles were removed in vacuo. The residue was purified by flash chromatography (SiO₂, hexanes/EtOAc, 2:1 to 100% EtOAc) to provide the desired product (139 mg, 14%) as a yellow solid. ¹H NMR (CDCl₃) δ 8.72 (1H, br s), 8.53 (1H, br s), 8.26 (1H, dd, J=1.6, 4.0 Hz), 7.77 (1H, br d, J=8.0 Hz), 7.70 (1H, dd, J=1.2, 4.8 Hz), 7.14 (1H, dd, J=3.6, 5.2 Hz), 6.99 (1H, d, J=3.6 Hz), 6.94 (1H, d, J=4.0 Hz), 5.45 (1H, br t, J=6.0 Hz), 4.84 (2H, d, J=6.0 Hz), 3.72 (3H, s). LC-MS: m/z=349 [M+H]⁺; RT=3.41 min.

### Example 7

### [7-Methyl-2-(1-pyridin-3-yl-ethylamino)-7H-pyrrolo[2,3-d]pyrimidin-4-yl]-thiophen-2-yl-methanone

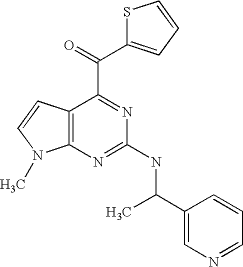

The title compound was prepared by the route outlined in scheme 1, following the same procedure described for Example 6. Purification by recrystallisation from EtOAc/hexanes. ¹H NMR (CDCl₃) δ 8.15 (1H, d, J=3.2 Hz), 7.80 (1H, br d, J=7.6 Hz), 7.70 (1H, dd, J=1.2, 4.8 Hz), 7.37 (1H, br s), 7.12 (1H, app t, J=4.4 Hz), 6.94 (1H, d, J=3.6 Hz), 6.88 (1H, d, J=3.2 Hz), 5.39 (2H, br s), 3.67 (3H, s), 1.66 (3H, d, J=6.0 Hz). LC-MS: m/z=364 [M+H]⁺; RT=3.31 min.

### Example 8

### {7-Methyl-2-[(pyridin-3-ylmethyl)-amino]-7H-pyrrolo[2,3-d]pyrimidin-4-yl}-(5-methyl-thiophen-2-yl)-methanone

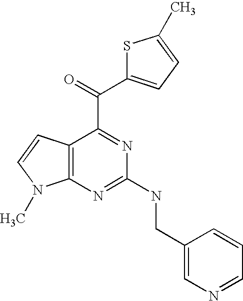

The title compound was prepared by the route outlined in scheme 1, following the same procedure described for Example 6. Purification by recrystallisation from EtOAc/hexanes. ¹H NMR (CDCl₃) δ 8.71 (1H, d, J=2.0 Hz), 8.52 (1H, dd, J=1.6, 4.8 Hz), 8.07 (1H, d, J=3.6 Hz), 7.76 (1H, app dt, J=2.0, 8.0 Hz), 7.25 (1H, dd, J=5.2, 7.6 Hz), 6.96 (1H, d, J=3.6 Hz), 6.91 (1H, d, J=3.6 Hz), 6.80 (1H, dd, J=1.2, 4.0 Hz), 5.46 (1H, brt, J=6.0 Hz), 4.83 (2H, d, J=6.0 Hz), 3.71 (3H, s), 2.53 (3H, s). LC-MS: m/z=363 [M+H]⁺, RT=3.54 min.

### Example 9

### {7-Methyl-2-[(pyridin-2-ylmethyl)-amino]-7H-pyrrolo[2,3-d]pyrimidin-4-yl}-(5-methyl-thiophen-2-yl)-methanone

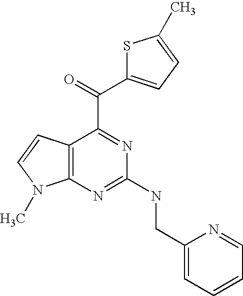

The title compound was prepared by the route outlined in scheme 1, following the same procedure described for Example 6. Purification by recrystallisation from EtOAc/hexanes. ¹H NMR (CDCl₃) δ 8.60 (1H, br d, J=5.2 Hz), 8.12 (1H, d, J=3.2 Hz), 7.63 (1H, app td, J=2.0, 7.6 Hz), 7.41 (1H, d, J=8.0 Hz), 7.18 (1H, dd, J=4.0, 7.6 Hz), 6.94 (1H, d, J=3.6 Hz), 6.92 (1H, d, J=3.6 Hz), 6.81 (1H, dd, J=1.2, 4.0 Hz), 5.94 (1H, app t, J=6.0 Hz), 4.95 (2H, d, J=6.0 Hz), 3.71 (3H, s), 2.54 (3H, s). LC-MS: m/z=364 [M+H]⁺; RT=3.63 min.

### Example 10

### {7-Methyl-2-[(pyridin-4-ylmethyl)-amino]-7H-pyrrolo[2,3-d]pyrimidin-4-yl}-(5-methyl-thiophen-2-yl)-methanone

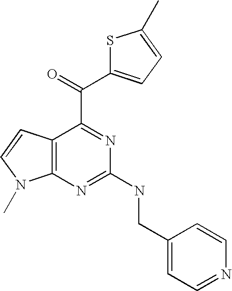

The title compound was prepared by the route outlined in scheme 1, following the same procedure described for Example 6. Purification by recrystallisation from EtOAc/hexanes. ¹H NMR (CDCl₃) δ 7.99 (1H, d, J=3.2 Hz), 7.49 (1H, br s), 6.96 (1H, d, J=3.6 Hz), 6.90 (1H, d, J=3.2 Hz), 6.78 (1H, d, J=3.6 Hz), 5.59 (1H, app t, J=6.0 Hz), 4.81 (1H, br d, J=6.0 Hz), 3.69 (3H, s), 2.51 (3H, s). LC-MS: m/z=364 [M+H]⁺; RT=3.17 min.

The compounds of Examples 11 to 22 were prepared as per the methodology described in scheme 2.

### Example 11

### 2,4-Dichloro-7-trimethylsilanylmethoxymethyl-7H-pyrrolo[2,3-d]pyrimidine

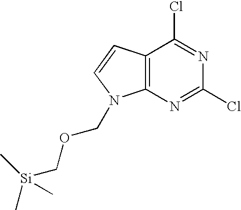

To a solution of Example 2 (571 mg, 3.037 mmol) and SEMCI (810 μL, 4.555 mmol) in 10 mL anhydrous THF was added portionwise NaH (182 mg, 4.555 mmol). After 1 h at RT, the reaction was quenched with water and the aqueous layer was extracted with EtOAc. The organics were combined, dried (MgSO₄), filtered and after evaporation of the volatiles, the residue was purified by flash chromatography (SiO₂, hexanes/EtOAc, 3:1) to provide the desired product (764 mg, 79%) as a yellow solid. ¹H NMR (CDCl₃) δ 7.71 (1H, d, J=3.6 Hz), 6.70 (1H, d, J=3.6 Hz), 5.64 (2H, s), 3.57 (2H, app t, J=8.0 Hz), 0.96 (2H, app t, J=8.0 Hz).

### Example 12

### (2-Chloro-7-trimethylsilanylmethoxymethyl-7H-pyrrolo[2,3-d]pyrimidin-4-yl)-thiophen-2-yl-methanone

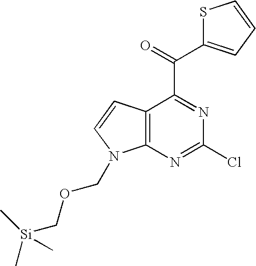

The title compound was prepared by the route outlined in scheme 2, following the same procedure described for Example 6. Purification by flash chromatography (SiO₂, hexanes/EtOAc, 5:1). ¹H NMR (CDCl₃) δ 8.55 (1H, dd, J=1.6, 3.6 Hz), 7.85 (1H, dd, J=1.2, 5.2 Hz), 7.56 (1H, d, J=3.2 Hz), 7.29 (1H, d, J=3.2 Hz), 7.27 (1H, dd, J=4.0, 5.2 Hz), 5.70 (2H, s), 3.60 (2H, app t, J=8.4 Hz), 0.00 (9H, s).

### Example 13

### (2-Chloro-7H-pyrrolo[2,3-d]pyrimidin-4-yl)-thiophen-2-yl-methanone

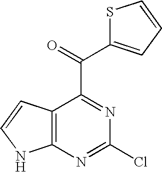

A solution of Example 12 (923 mg, 2.343 mmol) was treated with 9.4 mL of TBAF (1 M in THF, 4.686 mmol) and brought to reflux for 20 h. After cooling down to RT, the reaction mixture was poured over distilled water. The aqueous layer was extracted with EtOAc (2×50 mL). The organics were combined, dried (MgSO₄), filtered and the volatiles were removed in vacuo. The residue was purified by flash chromatography (SiO₂, hexanes/EtOAc, 2:1) to provide the desired product (137 mg, 22%) as a colourless solid. ¹H NMR (CDCl₃) δ 9.43 (1H, br s), 8.51 (1H, dd, J=1.2, 4.0 Hz), 7.82 (1H, dd, J=1.6, 5.2 Hz), 7.52 (1H, dd, J=2.0, 3.6 Hz), 7.27 (1H, dd, J=1.6, 4.8 Hz), 7.24 (1H, dd, J=4.0, 5.2 Hz). LC-MS: m/z=265 [M+H]⁺, RT=3.67 min; total run time=6 min

### Example 14

### {2-[(Pyridin-2-ylmethyl)-amino]-7H-pyrrolo[2,3-d]pyrimidin-4-yl}-thiophen-2-yl-methanone

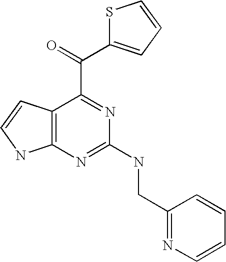

The title compound was prepared by the route outlined in scheme 2, following the same procedure described for Example 6. Purification by recrystallisation from EtOAc/hexanes. ¹H NMR (d₆-DMSO) δ 11.58 (1H, s), 8.61 (1H, d, J=2.0 Hz), 8.42 (1H, dd, J=1.6, 4.4 Hz), 8.29 (1H, br s), 8.08 (1H, dd, J=1.2, 4.8 Hz), 7.76 (1H, dt, J=2.0, 8.4 Hz), 7.64 (1H, br t, J=6.0 Hz), 7.32 (1H, dd, J=. 4.0, 8.0 Hz), 7.27 (1H, dd, J=2.0, 3.6 Hz), 7.24 (1H, app t, J=4.8 Hz), 6.68 (1H, dd, J=1.6, 3.6 Hz), 4.67 (2H, d, J=6.0 Hz). LC-MS: m/z=336 [M+H]⁺; RT=3.19 min; total run time=6 min

### Example 15

### {2-[(Pyridin-4-ylmethyl)-amino]-7H-pyrrolo[2,3-d]pyrimidin-4-yl}-thiophen-2-yl-methanone

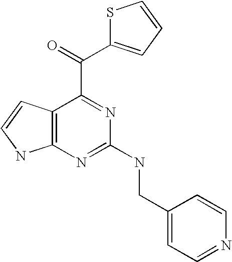

The title compound was prepared by the route outlined in scheme 2, following the same procedure described for Example 6. Purification by recrystallisation from EtOAc/hexanes. ¹H NMR (CDCl₃) δ 11.59 (1H, s), 8.52 (1H, d, J=4.4 Hz), 8.26 (1H, br s), 8.06 (1H, d, J=5.2 Hz), 7.70 (1H, td, J=1.6, 7.6 Hz), 7.61 (1H, br t, J=6.0 Hz), 7.36 (1H, d, J=7.2 Hz), 7.29-7.20 (2H, m), 6.69 (1H, dd, J=1.6, 3.6 Hz), 4.73 (2H, d, J=6.0 Hz). LC-MS: m/z=336 [M+H]⁺; RT=3.03 min.

### Example 16

### (2-Chloro-7-pentyl-7H-pyrrolo[2,3-d]pyrimidin-4-yl)-thiophen-2-yl-methanone

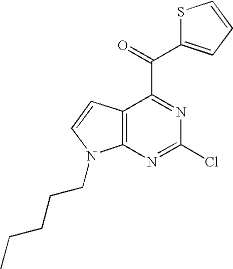

The title compound was prepared by the route outlined in scheme 2, following the same procedure described for Example 3. Purification by flash chromatography (SiO₂, hexanes/EtOAc, 3:1). ¹H NMR (CDCl₃) δ 8.42 (1H, dd, J=1.2, 4.0 Hz), 7.72 (1H, dd, J=1.2, 4.8 Hz), 7.31 (1H, d, J=3.6 Hz), 7.15 (1H, dd, J=3.6, 5.2 Hz), 7.10 (1H, d, J=3.6 Hz), 4.21 (2H, t, J=7.6 Hz), 1.80 (2H, app pentet, J=7.2 Hz), 1.32-1.18 (4H, m), 0.82 (3H, t, J=7.6 Hz). LC-MS: m/z=334 [M+H]⁺; RT=4.02 min.

### Example 17

### (7-Benzyl-2-chloro-7H-pyrrolo[2,3-d]pyrimidin-4-yl)-thiophen-2-yl-methanone

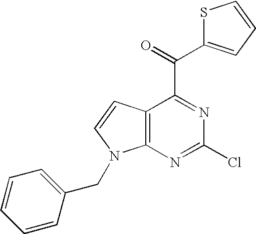

The title compound was prepared by the route outlined in scheme 2, following the same procedure described for Example 3. Purification by recrystallisation from EtOAc. ¹H NMR (CDCl₃) δ 8.49 (1H, dd, J=1.2, 4.0 Hz), 7.78 (1H, dd, J=1.2, 4.8 Hz), 7.34-7.16 (7H, m), 5.45 (2H, s). LC-MS: m/z=336 [M+H]⁺; RT=4.35 min.

### Example 18

### {7-Benzyl-2-[(pyridin-3-ylmethyl)-amino]-7H-pyrrolo[2,3-d]pyrimidin-4-yl}-thiophen-2-yl-methanone

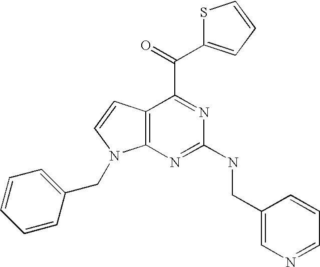

The title compound was prepared by the route outlined in scheme 2, following the same procedure described for Example 6. Purification by recrystallisation from Et₂O/EtOAc/hexanes. ¹H NMR (CDCl₃) δ 8.71 (1H, br s), 8.53 (1H, br s), 8.27 (1H, dd, J=1.6, 3.6 Hz), 7.73 (1H, br d, J=8.0 Hz), 7.70 (1H, dd, J=1.2, 5.2 Hz), 7.32-7.23 (5H, m), 7.18 (1H, dd, J=2.4, 6.8 Hz), 7.14 (1H, dd, J=4.0, 4.4 Hz), 6.99 (1H, d, J=3.6 Hz), 6.96 (1H, d, J=3.6 Hz), 5.48 (1H, brt, J=6.0 Hz), 5.29 (2H, s), 4.82 (2H, d, J=6.0 Hz). LC-MS: m/z=426 [M+H]⁺; RT=3.92 min; total run time=6 min

### Example 19

### {7-Pentyl-2-[(pyridin-3-ylmethyl)-amino]-7H-pyrrolo[2,3-d]pyrimidin-4-yl}-thiophen-2-yl-methanone

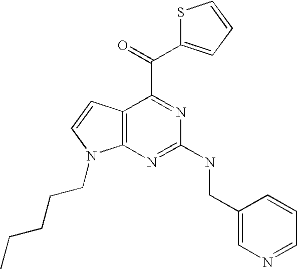

The title compound was prepared by the route outlined in scheme 2, following the same procedure described for Example 6. Purification by preparative mass directed LC. ¹H NMR (CDCl₃) δ 8.26 (1H, dd, J=1.6, 3.6 Hz), 7.77 (1H, br d, J=7.6 Hz), 7.70 (1H, dd, J=0.8, 5.2 Hz), 7.14 (1H, dd, J=4.0, 5.2 Hz), 7.01 (1H, d, J=3.2 Hz), 6.93 (1H, d, J=3.2 Hz), 5.46 (1H, br t, J=6.0 Hz), 4.83 (2H, d, J=6.0 Hz), 4.09 (2H, t, J=7.2 Hz), 1.79 (2H, app pentet, J=6.8 Hz), 1.37-1.23 (4H, m), 0.88 (3H, t, J=7.2 Hz). LC-MS: m/z=406 [M+H]⁺, RT=3.67 min.

### Example 20

### {7-Cyclopropylmethyl-2-[(pyridin-3-ylmethyl)-amino]-7H-pyrrolo[2,3-d]pyrimidin-4-yl}-thiophen-2-yl-methanone

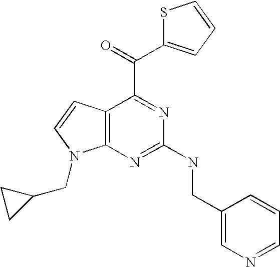

The title compound was prepared by the route outlined in scheme 2, following the same procedure described for Example 6. Purification by preparative mass directed LC. ¹H NMR (CDCl₃) δ 8.71 (1H, br s), 8.55 (1H, br s), 8.27 (1H, dd, J=1.2, 4.0 Hz), 7.77 (1H, d, J=7.6 Hz), 7.71 (1H, dd, J=1.6, 4.8 Hz), 7.16-7.13 (2H, m), 6.95 (1H, d, J=3.6 Hz), 5.46 (1H, app t, J=6.0 Hz), 4.83 (2H, d, J=6.0 Hz), 3.96 (2H, d, J=7.2 Hz), 0.92-0.82 (1H, m), 0.61-0.56 (2H, m), 0.39-0.35 (2H, m). LC-MS: m/z=390 [M+H]⁺; RT=3.56 min.

### Example 21

### {7-Propyl-2-[(pyridin-3-ylmethyl)-amino]-7H-pyrrolo[2,3-d]pyrimidin-4-yl}-thiophen-2-yl-methanone

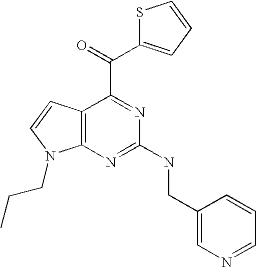

The title compound was prepared by the route outlined in scheme 2, following the same procedure described for Example 6. Purification by preparative mass directed LC. ¹H NMR (CDCl₃) δ 8.74 (1H, br s), 8.56 (1H, br s), 8.26 (1H, dd, J=1.2, 4.0 Hz), 7.77 (1H, d, J=8.0 Hz), 7.70 (1H, dd, J=0.8, 5.2 Hz), 7.14 (1H, dd, J=4.0, 4.4 Hz), 7.02 (1H, d, J=3.2 Hz), 6.93 (1H, d, J=3.6 Hz), 5.48 (1H, br t, J=6.0 Hz), 4.83 (2H, d, J=6.0 Hz), 4.07 (2H, t, J=7.6 Hz), 1.82 (2H, app hextet, J=6.8 Hz), 0.91 (3H, t, J=7.6 Hz). LC-MS: m/z=378 [M+H]⁺; RT=3.55 min.

### Example 22

### {7-Hydroxymethyl-2-[(pyridin-3-ylmethyl)-amino]-7H-pyrrolo[2,3-d]pyrimidin-4-yl}-thiophen-2-yl-methanone

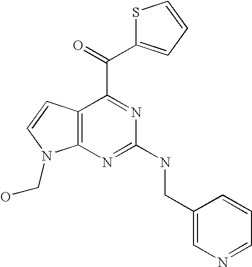

The title compound was prepared by the route outlined in scheme 2, following the same procedure described for Example 12. Purification by flash chromatography (SiO₂, gradient hexanes/EtOAc, 1:1 to EtOAc 100% to EtOAc/MeOH 95:5). ¹H NMR (d₅-DMSO) δ 8.63 (1H, d, J=1.6 Hz), 8.42 (1H, dd, J=1.6, 4.8 Hz), 8.25 (1H, br s), 8.09 (1H, dd, J=1.2, 4.8 Hz), 7.82-7.78 (2H, m), 7.37 (1H, d, J=3.6 Hz), 7.32 (1H, dd, J=4.0, 4.8 Hz), 7.23 (1H, app t, J=4.4 Hz), 6.73 (1H, d, J=3.6 Hz), 6.46 (1H, t, J=7.6 Hz), 5.46 (1H, d, J=7.2 Hz), 4.69 (1H, br d, J=6.0 Hz). LC-MS: m/z=378 [M+H]⁺; RT=3.55 min.

The compounds of Examples 23 to 30 were prepared as per the methodology described in scheme 3.

### Example 23

### 5-Bromo-2,4-dichloro-7-methyl-7H-pyrrolo[2,3-d]pyrimidine

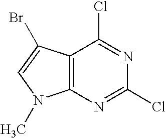

A solution of Example 3 (1.231 g, 3.093 mmol) in 2 mL anhydrous DMF was treated at 0° C. with a solution of NBS (1.193 g, 6.702 mmol) in 2 mL DMF. The ice-bath was removed and after 30 min, the reaction was quenched with an aqueous solution of sodium thiosulfate. The aqueous layer was extracted with EtOAc (2×). Organics were combined, dried (MgSO₄), filtered and the volatiles were removed in vacuo. The residue was purified by flash chromatography (50 g Isolute SiO₂ cartridge, gradient 100% hexanes to hexanes/EtOAc, 2:1) to provide the desired product (1.482 g, 86%) as a yellow solid. ¹H NMR (CDCl₃) δ 7.24 (1H, s), 3.84 (3H, s). LC-MS: m/z=281 [M+H]⁺; RT=3.51 min.

### Example 24

### (5-Bromo-2-chloro-7-methyl-7H-pyrrolo[2,3-d]pyrimidin-4-yl)-thiophen-2-yl-methanone

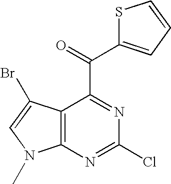

The title compound was prepared by the route outlined in scheme 3, following the same procedure described for Example 4. Purification by flash chromatography (SiO₂, hexanes/EtOAc, 4:1). ¹H NMR (CDCl₃) δ 7.85 (1H, dd, J=1.2, 4.8 Hz), 7.73 (1H, dd, J=1.2, 4.0 Hz), 7.34 (1H, s), 7.20-7.17 (1H, m), 3.89 (3H, s). LC-MS: m/z=357 [M+H]⁺; RT=3.59 min.

### Example 25

### {5-Bromo-7-methyl-2-[(pyridin-3-ylmethyl)-amino]-7H-pyrrolo[2,3-d]pyrimidin-4-yl}-thiophen-2-yl-methanone

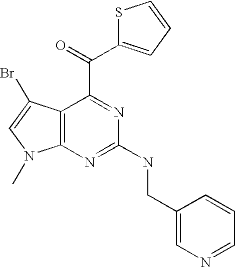

The title compound was prepared by the route outlined in scheme 3, following the same procedure described for Example 6. Purification by flash chromatography (SiO₂, gradient hexanes/EtOAc, 1:1 to EtOac 100%). ¹H NMR (CDCl₃) δ 8.68 (1H, br s), 8.52 (1H, br s), 7.76 (1H, dd, J=1.2, 4.8 Hz), 7.72 (1H, br d, J=8.0 Hz), 7.66 (1H, dd, J=1.6, 3.6 Hz), 7.12 (1H, dd, J=3.6, 4.8 Hz), 6.92 (1H, s), 5.58 (1H, brt, J=6.0 Hz), 4.73 (2H, d, J=6.0 Hz), 3.69 (3H, s). LC-MS: m/z=429 [M+H]⁺; RT=3.35 min.

### Example 26

### {7-Methyl-5-phenyl-2-[(pyridin-3-ylmethyl)-amino]-7H-pyrrolo[2,3-d]pyrimidin-4-yl}-thiophen-2-yl-methanone

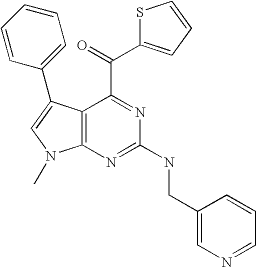

A solution of Example 25 (25 mg, 0.058 mmol), PhB(OH)₂ (10.6 mg, 0.087 mmol) and Pd(PPh₃)₄ (6.7 mg, 0.006 mmol) in 5 mL of THF/sat NaHCO₃(aq) (4:1) was refluxed for 2 h. The reaction mixture was diluted with sat NaHCO₃(aq) and EtOAc and the layers were separated. Organics were dried (MgSO₄), filtered and the volatiles removed in vacuo. The residue was purified by flash chromatography (SiO₂, EtOAc 100%) and recrystallisation from Et₂O/EtOAc/hexanes provided the desired product (3.9 mg, 16%). ¹H NMR (CDCl₃) δ 8.70 (1H, br d, J=2.0 Hz), 8.52 (1H, dd, J=1.6, 5.2 Hz), 7.74 (1H, br d, J=8.4 Hz), 7.64 (1H, dd, J=1.2, 4.8 Hz), 7.58 (1H, dd, J=1.2, 3.6 Hz), 7.20-7.13 (4H, m), 7.00 (1H, app t, J=4.4 Hz), 6.93 (1H, s), 5.55 (1H, brt, J=6.0 Hz), 4.76 (2H, d, J=6.0 Hz), 3.76 (3H, s). LC-MS: m/z=426 [M+H]⁺; RT=3.44 min.

### Example 27

### {5-(4-Methanesulfonyl-phenyl)-7-methyl-2-[(pyridin-3-ylmethyl)-amino]-7H-pyrrolo[2,3-d]pyrimidin-4-yl}-thiophen-2-yl-methanone

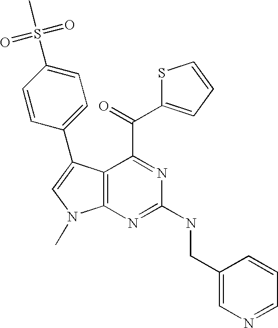

The title compound was prepared by the route outlined in scheme 3, following the same procedure described for Example 26. Purification by flash chromatography (SiO₂, gradient EtOAc 100% to EtOAc/MeOH, 95:5). ¹H NMR (CDCl₃) δ 8.71 (1H, br s), 8.54 (1H, br s), 7.77 (2H, d, J=8.4 Hz), 7.74 (1H, br s), 7.71 (1H, dd, J=1.6, 4.4 Hz), 7.69 (1H, dd, J=1.2, 4.0 Hz), 7.32 (2H, d, J=8.4 Hz), 7.06 (1H, dd, J=4.0, 4.8 Hz), 7.05 (1H, s), 5.72 (1H, br t, J=6.0 Hz), 4.77 (2H, d, J=6.0 Hz), 3.79 (3H, s), 3.06 (3H, s). LC-MS: m/z=504 [M+H]⁺, RT=2.33 min.

### Example 28

### {5-(4-Methoxy-phenyl)-7-methyl-2-[(pyridin-3-ylmethyl)-amino]-7H-pyrrolo[2,3-d]pyrimidin-4-yl}-thiophen-2-yl-methanone

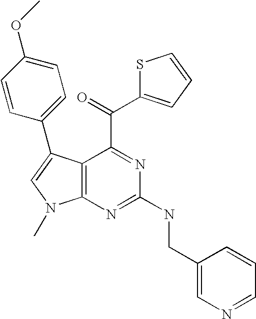

The title compound was prepared by the route outlined in scheme 3, following the same procedure described for Example 26. Purification by flash chromatography (SiO₂, EtOAc 100%). ¹H NMR (CDCl₃) δ 8.71 (1H, br s), 8.54 (1H, br s), 7.75 (1H, br d, J=8.0 Hz), 7.66 (1H, dd, J=0.8, 5.2 Hz), 7.57 (1H, dd, J=1.2, 3.6 Hz), 7.06 (2H, d, J=8.4 Hz), 7.00 (1H, dd, J=3.2, 5.2 Hz), 6.87 (1H, s), 6.74 (2H, d, J=9.2 Hz), 5.58 (1H, brt, J=6.0 Hz), 4.75 (2H, d, J=6.0 Hz), 3.77 (3H, s), 3.75 (3H, s). LC-MS: m/z=456 [M+H]⁺, RT=2.60 min.

### Example 29

### {5-(2-Methoxy-phenyl)-7-methyl-2-[(pyridin-3-ylmethyl)-amino]-7H-pyrrolo[2,3-d]pyrimidin-4-yl}-thiophen-2-yl-methanone

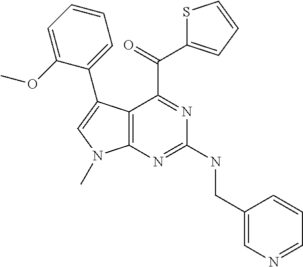

The title compound was prepared by the route outlined in scheme 3, following the same procedure described for Example 26. Purification by flash chromatography (SiO₂, EtOAc 100%). ¹H NMR (CDCl₃) δ 8.70 (1H, br s), 8.53 (1H, br s), 7.74-7.72 (2H, m), 7.59 (1H, dd, J=1.2, 4.8 Hz), 7.28 (1H, dd, J=2.0, 7.2 Hz), 727-7.22 (1H, m), 7.02 (1H, dd, J=4.0, 5.2 Hz), 6.97-6.93 (1H, m), 6.94 (1H, s), 6.69 (1H, d, J=7.2 Hz), 5.57 (1H, brt, J=6.0 Hz), 4.76 (2H, d, J=6.0 Hz), 3.74 (3H, s), 3.17 (3H, s). LC-MS: m/z=456 [M+H]⁺; RT=3.07 min.

### Example 30

### {5-(3-Methoxy-phenyl)-7-methyl-2-[(pyridin-3-ylmethyl)-amino]-7H-pyrrolo[2,3-d]pyrimidin-4-yl}-thiophen-2-yl-methanone

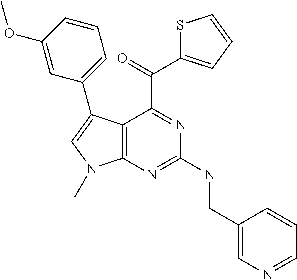

The title compound was prepared by the route outlined in scheme 3, following the same procedure described for Example 26. Purification by flash chromatography (SiO₂, EtOAc 100%). ¹H NMR (CDCl₃) δ 8.70 (1H, br s), 8.51 (1H, br d, J=3.6 Hz), 7.74 (1H, d, J=8.4 Hz), 7.63 (1H, dd, J=0.8, 5.2 Hz), 7.55 (1H, dd, J=0.8, 4.0 Hz), 7.25 (1H, dd, J=4.8, 8.0 Hz), 7.10 (1H, app t, J=6.0 Hz), 6.99 (1H, dd, J=4.0, 4.8 Hz), 6.93 (1H, s), 6.75-6.74 (1H, m), 6.73-6.72 (1H, m), 6.70-6.68 (1H, m), 5.57 (1H, br t, J=6.0 Hz), 4.75 (2H, d, J=6.0 Hz), 3.75 (3H, s), 3.64 (3H, s). LC-MS: m/z=456 [M+H]⁺; RT=3.10 min.

The compounds of Examples 31 to 36 were prepared as per the methodology described in scheme 4.

### Example 31

### 7-Benzenesulfonyl-2,4-dichloro-7H-pyrrolo[2,3-d]pyrimidine

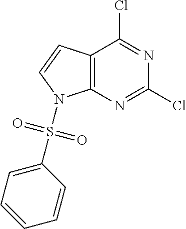

A solution of Example 2 (566 mg, 3.01 mmol) in 5 mL anhydrous THF at 0° C. was treated with NaH (180.6 mg, 4.515 mmol) portionwise. After 50 min at 0° C., the reaction mixture was treated with PhSO₂Cl (691.1 mg, 3.913 mmol). After 1.5 h at RT, the reaction mixture was quenched with sat NH₄Cl(aq) (30 mL). The aqueous layer was extracted with EtOAc. Organics were combined, dried (MgSO₄), filtered and the volatiles were removed in vacuo. The residue was purified by flash chromatography (20 g (solute SiO₂ cartridge, gradient hexanes 100% to hexanes/EtOAc, 2:1) to provide the desired product (910 mg, 92%) as a colourless foam. ¹H NMR (CDCl₃) δ 8.25-8.22 (2H, m), 7.76 (1H, d, J=4.0 Hz), 7.67 (1H, dt, J=2.0, 10.0 Hz), 7.60-7.56 (2H, m), 6.69 (1H, d, J=4.0 Hz). LC-MS: m/z=328 [M+H]⁺; RT=3.42 min.

### Example 32

### 7-Benzenesulfonyl-6-bromo-2,4-dichloro-7H-pyrrolo[2,3-d]pyrimidine

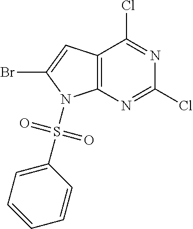

A solution of LDA in THF, prepared at 0° C. by addition of n-BuLi (2.5 M in hexanes, 2.5 mL, 6.378 mmol) to a solution of i-Pr₂NH (980 μL, 6.932 mmol) in 5 mL of anhydrous THF, cooled to −78° C. was treated with a solution of Example 31 (910 mg, 2.773 mmol) in 10 mL THF. After 30 min, the reaction mixture was treated with a solution of 1,2-dibromo-tetrachloroethane (2.709 g, 8.319 mmol) in 10 mL THF. After 2 h at −78° C., the reaction mixture was quenched with sat NH₄Cl(aq) (20 mL). The aqueous layer was extracted with EtOAc (2×). Organics were combined, dried (MgSO₄), filtered and the volatiles were removed in vacuo. The residue was purified by flash chromatography (50 g Isolute SiO₂ cartridge, gradient hexanes 100% to hexanes/EtOAc, 4:1) to provide the desired product (805 mg, 71%) as a colourless solid. ¹H NMR (CDCl₃) δ 8.26-8.23 (2H, m), 7.68 (1H, dt, J=1.2, 7.6 Hz), 7.60-7.56 (2H, m), 6.81 (1H, s).

### Example 33

### 6-Bromo-2,4-dichloro-7H-pyrrolo[2,3-c]pyrimidine

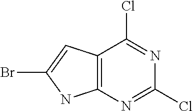

A solution of Example 32 (805 mg, 1.977 mmol) in 20 mL of anhydrous THF was treated with KOtBu (1.1 g, 9.888 mmol). After 18 h at RT, the reaction was quenched with saturated NaHCO₃. The aqueous layer was extracted with EtOAc (2×). Organics were combined, dried (MgSO₄), filtered and the volatiles were removed in vacuo. The residue was purified by flash chromatography (50 g Isolute SiO₂ cartridge, gradient hexanes 100% to hexanes/EtOAc, 2:1) to provide the desired product (334 mg, 63%) as a colourless solid. ¹H NMR (CDCl₃) δ 9.64 (1H, br s), 6.70 (1H, d, J=2.0 Hz). LC-MS: m/z=267 [M+H]⁺, RT=3.05 min.

### Example 34

### 6-Bromo-2,4-dichloro-7-methyl-7H-pyrrolo[2,3-c]pyrimidine

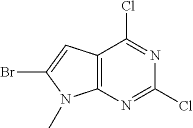

A solution of 6-bromo-2,4-dichloro-7H-pyrrolo[2,3-d]pyrimidine, prepared in Example 33 (334 mg, 1.251 mmol) in 10 mL of anhydrous THF, cooled to 0° C. was treated with NaH (75 mg g, 1.877 mmol). After 30 min at 0° C., the reaction mixture was treated with MeI (235 μL, 3.753 mmol). After 21 h at RT, the reaction was quenched with distilled water. The aqueous layer was extracted with EtOAc (2×). Organics were combined, dried (MgSO₄), filtered and the volatiles were removed in vacuo. The residue was purified by flash chromatography (20 g Isolute SiO₂ cartridge, gradient hexanes 100% to hexanes/EtOAc, 4:1) to provide the desired product (259 mg, 74%) as a colourless solid. ¹H NMR (CDCl₃) δ 6.73 (1H, s), 3.83 (3H, s). LC-MS: m/z 281 [M+H]⁺; RT=3.35 min.

### Example 35

### (6-Bromo-2-chloro-7-methyl-7H-pyrrolo[2,3-d]pyrimidin-4-yl)-thiophen-2-yl-methanone

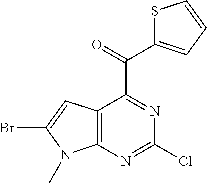

The title compound was prepared by the route outlined in scheme 4, following the same procedure described for Example 4. Purification by flash chromatography (20 g Isolute SiO₂ cartridge, gradient hexanes 100% to hexanes/EtOAc, 5:1). ¹H NMR (CDCl₃) δ 8.51 (1H, dd, J=1.6, 4.0 Hz), 7.81 (1H, dd, J=1.6, 5.2 Hz), 7.35 (1H, s), 7.23 (1H, dd, J=4.0, 5.2 Hz), 3.88 (3H, s). LC-MS: m/z=357 [M+H]⁺, RT=3.68 min.

### Example 36

### {6-Bromo-7-methyl-2-[(pyridin-3-ylmethyl)-amino]-7H-pyrrolo[2,3-d]pyrimidin-4-yl}-thiophen-2-yl-methanone

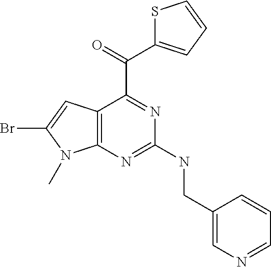

The title compound was prepared by the route outlined in scheme 4, following the same procedure described for Example 6. Purification by flash chromatography (20 g Isolute SiO₂ cartridge, gradient hexanes/EtOAc, 1:1 to EtOAc 100%). ¹H NMR (CDCl₃) δ 8.26 (1H, dd, J=0.8, 4.0 Hz), 7.78 (1H, br d, J=7.6 Hz), 7.70 (1H, dd, J=1.6, 4.8 Hz), 7.14 (1H, dd, J=4.0, 5.2 Hz), 7.09 (1H, s), 5.47 (1H, brt, J=6.0 Hz), 4.84 (2H, d, J=6.0 Hz). LC-MS: m/z=430 [M+H]⁺; RT=3.21 min.

The compounds of Examples 37 to 41 were prepared as per the methodology described in scheme 5.

### Example 37

### 2,4,6-Trichloro-7H-pyrrolo[2,3-d]pyrimidine

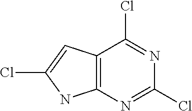

A suspension of commercially available 6-amino-1H-pyrimidine-2,4-dione (20.068 g, 157.89 mmol) in 100 mL DMF was treated with ethyl bromoacetate (17.5 mL, 157.89 mmol) and brought to reflux. After 4.5 h, the reaction mixture was cooled to 0° C. and the orange solid was filtered, rinsed with Et₂O (2×) and dried in vacuo. The highly insoluble orange solid was subsequently treated with PhPOCl₂ (120 mL) and brought to reflux. After 20 h at reflux, the reaction mixture was poured carefully over crushed ice (external cooling bath may be required) and filtered through Celite. The aqueous layer was extracted with EtOAc (2×) and the organics were combined, washed with sat NaHCO₃/NaHCO₃(s), dried (MgSO₄) and filtered. The residue was purified by flash chromatography (50 g Isolute SiO₂ cartridge, gradient hexanes 100% to hexanes/EtOAc, 2:1) to provide the desired product (811 mg, 2%) as a colourless solid. ¹H NMR (CDCl₃) δ 10.31 (1H, br s), 6.57 (1H, d, J=1.6 Hz). LC-MS: m/z=223 [M+H]⁺, RT=2.99 min.

### Example 38

### 2,4,6-Trichloro-7-methyl-7H-pyrrolo[2,3-d]pyrimidine

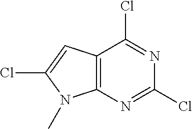

The title compound was prepared by the route outlined in scheme 5, following the same procedure described for Example 3. Purification by flash chromatography (50 g Isolute SiO₂ cartridge, gradient hexanes 100% to hexanes/EtOAc, 2:1). ¹H NMR (CDCl₃) δ 6.58 (1H, s), 3.81 (3H, s). LC-MS: m/z=236 [M+H]⁺, RT=3.25 min.

### Example 39

### (2,6-Dichloro-7-methyl-7H-pyrrolo[2,3-d]pyrimidin-4-yl)-thiophen-2-yl-methanone

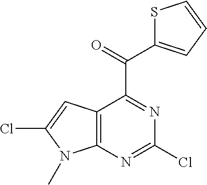

The title compound was prepared by the route outlined in scheme 5, following the same procedure described for Example 4. Purification by flash chromatography (50 g Isolute SiO₂ cartridge, gradient hexanes 100% to hexanes/EtOAc, 3:1). ¹H NMR (CDCl₃) δ 8.51 (1H, dd, J=1.2, 4.0 Hz), 7.81 (1H, dd, J=0.8, 5.2 Hz), 7.23 (1H, dd, J=4.0, 5.2 Hz), 7.21 (1H, s), 3.86 (3H, s). LC-MS: m/z=312 [M−H]⁻; RT=3.64 min.

### Example 40

### {6-Chloro-7-methyl-2-[(pyridin-3-ylmethyl)-amino]-7H-pyrrolo[2,3-d]pyrimidin-4-yl}-thiophen-2-yl-methanone

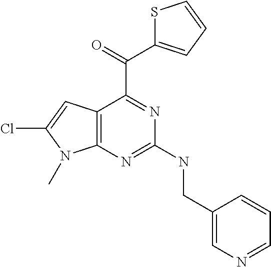

The title compound was prepared by the route outlined in scheme 5, following the same procedure described for Example 6. Purification by preparative mass directed LC. ¹H NMR (CDCl₃) δ 8.26 (1H, dd, J=1.2, 3.6 Hz), 7.81 (1H, br d, J=7.6 Hz), 7.70 (1H, dd, J=1.6, 4.8 Hz), 7.14 (1H, dd, J=4.0, 4.4 Hz), 6.94 (1H, s), 5.51 (1H, br t, J=6.0 Hz), 4.85 (2H, d, J=6.0 Hz). LC-MS: m/z=384 [M+H]⁺; RT=3.16 min.

### Example 41

### Thiophene-2-carboxylic acid [7-methyl-2-[(pyridin-3-ylmethyl)-amino]-4-(thiophene-2-carbonyl)-7H-pyrrolo[2,3-d]pyrimidin-6-yl]-amide

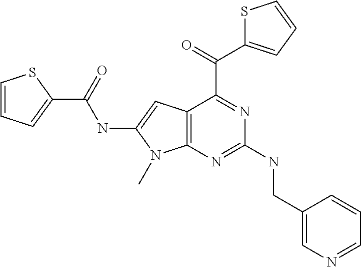

To a pre-stirred solution of Pd(OAc)₂ (1.4 mg, 0.006 mmol) and XantPhos (7 mg, 0.012 mmol) in 1 mL THF was added a solution of Example 40 (23.2 mg, 0.060 mmol), NaOtBu (8.3 mg, 0.087 mmol) and thiophene-2-carboxamide (11.5 mg, 0.091 mmol) in 1 mL THF. The reaction mixture was irradiated by microwaves at 130° C. for 10 min. The reaction was diluted with brine and EtOAc. Organics were dried (MgSO₄), filtered and the volatiles were removed in vacuo. The residue was filtered through a short plug of SiO₂ and purified by preparative mass-directed LC to provide the desired product (2.3 mg, 8%) as a yellow solid. ¹H NMR (CD₃OD) δ 8.64 (1H, br s), 8.40 (1H, br s), 8.25 (1H, br d, J=3.6 Hz), 7.95-7.92 (2H, m), 7.87 (1H, dd, J=1.2, 4.8 Hz), 7.79 (1H, dd, J=1.2, 5.2 Hz), 7.39 (1H, dd, J=4.4, 8.0 Hz), 7.22 (1H, dd, J=4.4, 4.8 Hz), 7.17 (1H, dd, J=4.0, 5.2 Hz), 6.78 (1H, s), 3.62 (3H, s). LC-MS: m/z=475 [M+H]⁺; RT=3.07 min.

### General Procedures

All reagents obtained from commercial sources were used without further purification. Anhydrous solvents were obtained from commercial sources and used without further drying. Flash chromatography was performed with pre-packed silica-gel cartridges (Strata Si-1; 61 Å, Phenomenex, Cheshire, UK or IST Flash II, 54 Å, Argonaut, Hengoed, UK). Thin layer chromatography was conducted with 5×10 cm plates coated with Merck Type 60 F₂₅₄ silica-gel. Microwave heating was performed with a Biotage Initiator™2.0 instrument.

The compounds of the present invention were characterized by liquid chromatography-mass spectroscopy (LC-MS) using the following method.

Instrument: Waters 2695 pump and 2700 sample manager
Waters ZQ2000, M/z range 100 to 900 amu

Column: Gemini 5 μm, C18 110A, 30 mm×2 mm from Phenomenex. Pt no 00A-4435-B0
Temperature: Ambient

Mobile Phase: A—Water+10 mMol/ammonium formate+0.04% (v/v) formic acid at pH ca 3.5
B—100% Acetonitrile+0.04% (v/v) formic acid
Injection Volume 10 uL
Gradient:

| Time (min) | Solvent A (%) | Solvent B (%) | Flow (cm³min⁻¹) |
|---|---|---|---|
| −0.8 (Equil) | 95 | 5 | 1.0 |
| 0 | 95 | 5 | 0.8 |
| 0.25 | 95 | 5 | 0.8 |
| 2.50 | 5 | 95 | 0.8 |
| 4.0 | 5 | 95 | 0.8 |
| 5 | 5 | 95 | 1.0 |
| 5.2 | 95 | 5 | 1.0 |

Detection: UV detection from 220 to 400 nm (1:3 split MS to UV)

Nuclear magnetic resonance (NMR) analysis was performed with a Bruker DPX400 spectrometer and proton NMR spectra were measured at 400 MHz. The spectral reference was the known chemical shift of the solvent. Proton NMR data is reported as follows: chemical shift (δ) in pμm, followed by the integration, the multiplicity (where s=singlet, d=doublet, t=triplet, q=quartet, p=pentet, m=multiplet, dd=doublet of doublets and br=broad), and the coupling constant rounded to the nearest 0.1 Hz.

Some compounds of the invention were purified by preparative HPLC. These were performed on a Waters FractionLynx MS autopurification system, with a Gemini® 5 μm C18(2), 100 mm×20 mm i.d. column from Phenomenex, running at a flow rate of 20 cm³ min⁻¹ with UV diode array detection (210-400 nm) and mass-directed collection. Gradients used for each compound are shown in Table 1.

At pH 4: solvent A=10 mM ammonium acetate in HPLC grade water+0.08% v/v formic acid. Solvent B=95% v/v HPLC grade acetonitrile+5% v/v solvent A+0.08% v/v formic acid.

At pH 9: solvent A=10 mM ammonium acetate in HPLC grade water+0.08% v/v ammonia solution. Solvent B=95% v/v HPLC grade acetonitrile+5% v/v solvent A+0.08% v/v ammonia solution.

The mass spectrometer was a Waters Micromass ZQ2000 spectrometer, operating in positive or negative ion electrospray ionisation modes, with a molecular weight scan range of 150 to 1000.

TABLE 1
Preparative HPLC gradients

| | % Solvent B for Example No. | | | | | |
|---|---|---|---|---|---|---|
| | | 6, 8, 11, | | | | |
| Time | 4, 5 | 12, 15-18, | 19, 20 | 2, 3, 9, | | |
| (min) | and 14 | 21 and 26 | and 23 | 10 and 13 | 24 | 27 |
| 0.0 | 5 | 5 | 5 | 5 | 5 | 5 |
| 0.5 | 6 | 15 | 15 | 30 | 10 | 25 |
| 7.0 | 25 | 30 | 40 | 40 | 20 | 50 |
| 7.5 | 95 | 95 | 95 | 95 | 95 | 95 |
| 9.5 | 95 | 95 | 95 | 95 | 95 | 95 |
| 10 | 5 | 5 | 5 | 5 | 5 | 5 |

IUPAC chemical names were generated using AutoNom Standard.

### Assay Description

The use of a Fluorometric Imaging Plate Reader (FLIPR) to measure calcium flux in Adenosine-receptor expressing cells is a well-established technique. In this assay calcium flux is triggered by receptor activation and measured through the fluorescence of an incorporated calcium-sensitive dye. The potencies shown were determined using expressed human adenosine A_{2B} receptors in mammalian cell lines. Selectivity values were obtained by using mammalian cell lines expressing the human adenosine A₁, A_{2A} and A₃ receptors. Compound potency was determined from dose response curves and are reported as IC₅₀ values.

The compounds tested in the above assay were assigned to one of two activity ranges, namely A=IC₅₀<500 nM, or B=IC₅₀>500 nM, as indicated in Table 2 below.

TABLE 2

| Example | Activity |
|---|---|
| 6 | A |
| 7 | A |
| 8 | A |
| 9 | A |
| 10 | B |
| 13 | B |
| 14 | B |
| 17 | B |
| 18 | B |
| 19 | B |
| 20 | B |
| 21 | B |
| 25 | B |
| 26 | B |
| 27 | B |
| 28 | B |
| 29 | B |
| 35 | A |
| 39 | A |
| 40 | B |

1. A compound of formula (I) or a pharmaceutically acceptable salt, hydrate or solvate thereof: 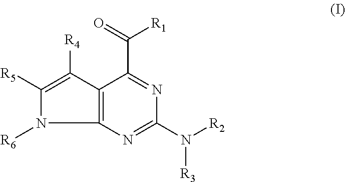
wherein
R₁ is optionally substituted aryl or an optionally substituted monocyclic heteroaryl group having 5 or 6 ring atoms;
R₂ and R₃ are independently selected from hydrogen, or optionally substituted C₁-C₆ alkyl, C₁-C₆ alkoxy-(C₁-C₆)-alkyl, C₃-C₈ cycloalkyl, aryl, heteroaryl, aryl-(C₁-C₆)-alkyl, or heteroaryl-(C₁-C₆)-alkyl;
R₄ and R₅ are independently selected from hydrogen, optionally substituted C₁-C₆ alkyl, optionally substituted aryl, aryl-(C₁-C₆)-alkyl optionally substituted in the ring part thereof, —NHR₇, —N(—R₈)—R₉, —NH—(C═O)—R₁₀, —(C═O)—NH—R₁₁, —(C═O)—O—R₁₂, or halo;
R₆ is hydrogen, C₁-C₆ alkyl, aryl-(C₁-C₆)-alkyl, —(C═O)—NH—R₁₃, —(C═O)—R₁₄, aryl, heteroaryl, hydroxy-(C₁-C₆)-alkyl, or C₃-C₈ cycloalkyl-alkyl; and
R₇, R₈, R₉, R₁₀, R₁₁, R₁₂, R₁₃ and R₁₄ are independently selected from C₁-C₆ alkyl, aryl, aryl-(C₁-C₆)-alkyl and heteroaryl.

2. A compound as claimed in claim 1 wherein R₁ is an optionally substituted monocyclic heteroaryl group having 5 or 6 ring atoms.

3. A compound as claimed in claim 1 wherein R₁ is thienyl optionally substituted by fluoro, chloro, bromo, cyano, methyl, trifluoromethyl, ethyl, hydroxyl, hydroxymethyl, or hydroxyethyl.

4. A compound as claimed in claim 1 wherein R₂ and R₃ are independently selected from hydrogen, or heteroaryl-(C₁-C₆)-alkyl optionally substituted by fluoro, chloro, bromo, cyano, methyl, trifluoromethyl, ethyl, hydroxyl, hydroxymethyl, or hydroxyethyl.

5. A compound as claimed in claim 1 wherein R₄ and R₅ are independently selected from hydrogen, halo, optionally substituted aryl, or heteroarylcarbonylamino.

6. A compound as claimed in claim 5 wherein R₄ and R₅ are independently selected from hydrogen, chloro, bromo, phenyl, 2-methoxyphenyl, 3-methoxyphenyl, 4-methoxyphenyl, 4-methylsulphonylphenyl, or 2-thienylcarbonylamino.

7. A compound as claimed in claim 1 wherein R₅ is —N(—R₁₅)—R₁₆, and wherein R₁₅ and R₁₆ are independently selected from hydrogen or C₁-C₆ alkyl.

8. A compound as claimed in claim 1 wherein R₆ is hydrogen, C₁-C₆ alkyl, aryl-(C₁-C₆)-alkyl, hydroxy-(C₁-C₆)-alkyl, or C₃-C₈ cycloalkyl-alkyl.

9. A compound as claimed in claim 8 wherein R₆ is hydrogen, methyl, n-propyl, n-pentyl, benzyl, hydroxymethyl, or cyclopropylmethyl.

10. A compound of formula (II) or a pharmaceutically acceptable salt, hydrate or solvate thereof: 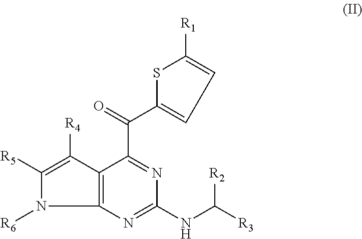
wherein
R₁ and R₂ are independently selected from hydrogen or C₁-C₆ alkyl;
R₃ is 2-, 3-, or 4-pyridyl;
R₄ and R₅ are independently selected from hydrogen, halo, optionally substituted aryl, or heteroarylcarbonylamino; and
R₆ is hydrogen, C₁-C₆ alkyl, aryl-(C₁-C₆)-alkyl, hydroxy-(C₁-C₆)-alkyl, or C₃-C₈ cycloalkyl-alkyl.

11. A compound as claimed in claim 10 wherein R₄ and R₅ are independently selected from hydrogen, chloro, bromo, phenyl, 2-methoxyphenyl, 3-methoxyphenyl, 4-methoxyphenyl, 4-methylsulphonylphenyl, or 2-thienylcarbonylamino

12. A compound as claimed in claim 10 wherein R₆ is hydrogen, methyl, n-propyl, n-pentyl, benzyl, hydroxymethyl, or cyclopropylmethyl.

13. A pharmaceutical composition comprising a compound as claimed in claim 1 and a pharmaceutically acceptable carrier.

14. (canceled)

15. A method of treating a disorder mediated by the adenosine A_{2B} receptor comprising the administration to a subject suffering such a disorder an effective amount of a compound as claimed in claim 1.

16. The method as claimed in claim 15 wherein the disorder mediated by the adenosine A_{2B} receptor is nociception, asthma, COPD, an inflammatory disorder, diabetes, diabetic retinopathy or cancer.
